(19) United States
(12) Patent Application Publication
(10) Pub. No.: US 2014/0180208 A1
(43) Pub. Date: Jun. 26, 2014

(54) SAFE NEEDLE DEVICE FOR SYRINGES

(71) Applicants: Dietrich Walter Grabis, San Rafael, CA (US); Songneng Luo, Liusha Town (CH)

(72) Inventors: Dietrich Walter Grabis, San Rafael, CA (US); Songneng Luo, Liusha Town (CH)

(73) Assignee: International Technology Group, San Rafael, CA (US)

(21) Appl. No.: 13/723,258

(22) Filed: Dec. 21, 2012

(51) Int. Cl. A61M 5/34; A61M 5/50

(52) U.S. Cl. CPC A61M 5/34; A61M 5/50
USPC 604/110

(57) ABSTRACT

A needle stick safety syringe includes a plunger where a concentric coil spring is compressed against the inside front end of the plunger cavity and retained there by a stop which is incorporated at the distal end of the inner cylindrical plunger body. The stop is released when the plunger is pushed all the way forward, which causes the plunger to be engaged with the needle base through interconnecting means. The release is activated by holding the syringe in one hand allowing the needle to be retracted into the cylindrical plunger cavity and to prevent reuse.

### INTRODUCTION

The present invention is directed in general to a safe needle device for syringes as used in the medical industry and hospitals. The “Improvement Patent” (classified as a UTILITY PATENT, according to the USPTO) application presents two significant improvements which are based upon numerous marketability tests (see Commercial Feasibility after the Background section below) conducted over the past several years and after FDA 510(k) approval to market the invention, issued under K010477. The two improvements are:
a) reducing the bulkiness of the device as expressed by Vanderbilt University's Medical School of Nursing, Nashville, Tenn. and other potential users.
b) providing an ergonomically more convenient location and use of the retraction release.

### BACKGROUND OF THE INVENTION

With the advent of AIDS and other highly infectious diseases, syringes and scalpels and other sharps devices have caused numerous injuries and infections to administering medical staff. Protective devices have been introduced to the market after the U.S. legislation in 1998, but consisted of needle covers that still left part of the needle exposed after use and thereby subjecting the clinician to exposure to contaminated blood. These devices are still on the market, but are not user friendly and safe. For example, a nurse must use two hands to prepare a patient for an injection. One hand is used to clean the area to be injected with a disinfectant while the other hand is used to inject the needle into the tissue. After removing the needle from the patient, the administering nurse must press on the spot of injection and hold the syringe in the other hand. After pressing the spot is complete (to stop the flow of blood), the nurse, in prior protective devices, then uses that hand to twist a cover over the syringe or sharp. That requires two hands during which time the needle may drip blood onto the patient or the nurse where contamination is inevitable. In addition, the maneuvering to accomplish the foregoing may cause an inadvertent puncturing of the nurse's own skin, thus inviting infection. One such device is Badger U.S. Pat. No. 5,885,257, which is a very cumbersome releasable retaining device to retract the needle where it will not cause harm.

In this Improvement Patent filing the above statements remain unchanged, but the ease of handling the device during administering medication by a clinician is greatly improved and so is increased safety to the patient and clinician. Two mechanically retracting safety syringes came on the market, i.e. the Integra, marketed by BD (Becton Dickenson) of Franklin, N.J., U.S. Pat. No. 5,632,733 (Shaw) and U.S. Pat. No. 7,351,224 (Shaw) and the Vanish Point, marketed by RTI (Retractable Technology, Inc.) of Houston, Tex., and U.S. Pat. No. 6,090,077 (Shaw, 7/2000). While the spring-operated needle retraction is the same in both devices (BD and RTI), they both have their compression springs located in the front end of the syringe whereas the Grabis invention has the spring located inside the plunger. In addition, the Vanish Point does not have a Luer-Lock and, thus, does not permit interchange-ability of needles prior to use. The Integra is hard to push to cause retraction which is a detriment for single hand operation because it can cause significant patient discomfort especially when the device is triggered while the needle is still in the patient. The spring assembly in the Integra (BD) can easily be removed after retraction; it falls apart.

In U.S. Pat. No. 5,053,010, McGary states in claim 1. “. . . a cutting tip configured inside said sealing member . . . ” in claim 2 he states “. . . said cutting tip is a beveled knife”.

The Grabis invention does not use any cutting tip. It uses a mechanically inter-locking tip-lock. In U.S. Pat. No. 5,211,629, Pressly (10/1991) states “Positioned between sacrificial supports in the needle assembly and the barrel is a deformable base and . . . the plunger has a rupturable boot”. The McGary and Pressly inventions are quite a departure from the Grabis invention.

After extensive marketability testing and commercial feasibility evaluation, the Grabis invention was made faster in retraction, smaller in the outer diameter, lighter and cheaper to manufacture.

### OBJECT AND SUMMARY OF THE IMPROVEMENTS

It is a general object of the improvements to the original invention, as shown in patent U.S. Pat. No. 6,322,540 B1 to provide a further improved retractable safety needle device for syringes.

In accordance with the above object, there is provided a needle-stick safety syringe comprising a tubular body, cylindrical plunger, and extended needle at the distal end of the tubular body. Said needle to be removable, through attachment means, in order to be replaced with a different size needle if so desired or required based upon patient needs.

Latch means, permitting the retraction of the needle, provide the interconnection between the distal end of the plunger and the needle base. Said needle base having a cylindrical body inside a cylindrical adapter means. Said adapter having a male thread at its distal end in form of a ring that comprises a single male thread that provides means to engage it into a mating female thread. Said female thread is at the distal end of the syringe body. Said cylindrical body inside said cylindrical adapter performs a dual function. It provides the means to firmly encase the base of the needle and is shaped on the opposite end to provide means to engage the base of the plunger.

The coil spring is placed inside said plunger behind a plug that provides means to interconnect said plunger with said needle base. Said stop inside said plunger is limiting further expansion of said spring after said needle retraction is activated.

### BRIEF DESCRIPTION OF THE DRAWINGS

FIG. 1 is a cross-sectional view of a syringe embodying the improved invention showing its basic standard syringe construction with a female Luer-Lock connection and vapor lock.

FIG. 2 is a cross-sectional view of the needle base assembly with needle connection means to allow the needle to disappear inside the plunger after activation.

FIG. 3 shows a cross-sectional view of the retracted needle inside the plunger and the compressed spring in the retention means at the forward end of the plunger.

FIG. 4 shows a side view of the syringe.

### DETAILED DESCRIPTION OF THE PREFERRED EMBODIMENT

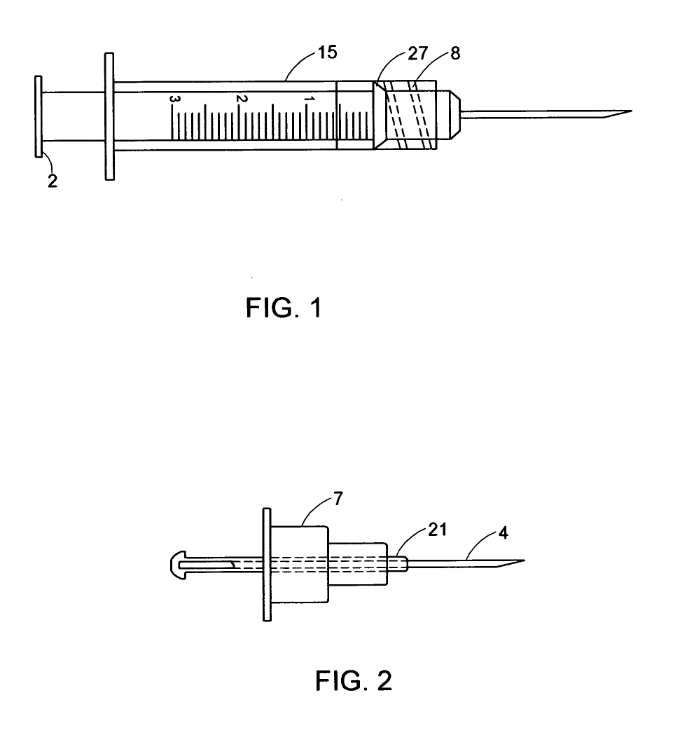

Referring now to FIG. 1, a typical commercial syringe that has a 3 ml capacity and is illustrated with a tubular body 15 and plunger 1 that slides within the tubular body operates in the standard manner. The tubular syringe body 15 has a flange 2 at its proximal end and an extended female Luer-Lock screw-on connector at its distal end 8 which is an integral part of the syringe body 15.

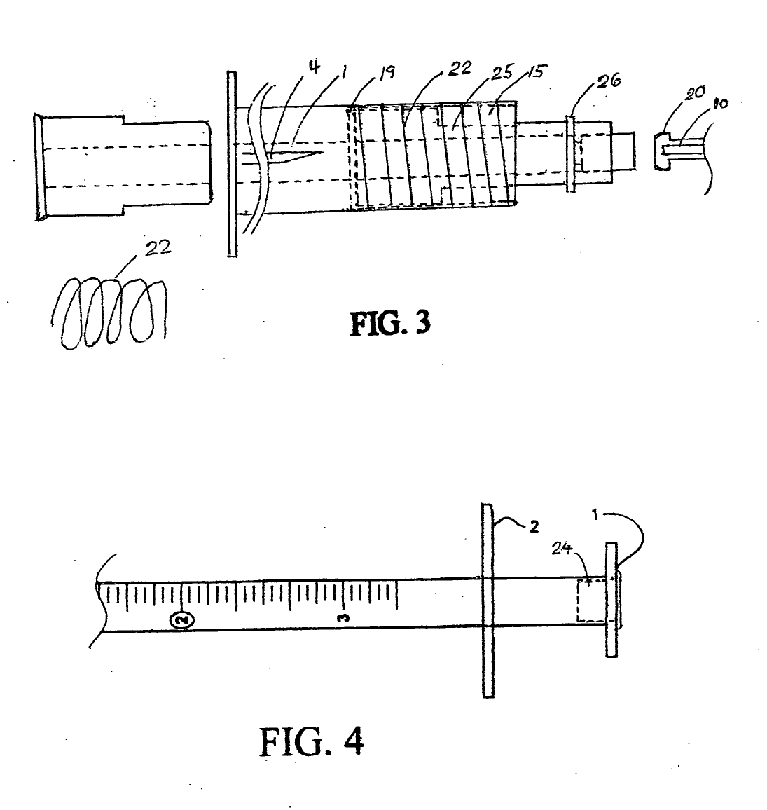

FIG. 2 shows a needle base assembly 3 providing means to permit extracting the needle 4 from a sleeve 7. The needle 4 is encased in a bonding plastic material at its distal end, as is done with all syringes whether they are safety syringes or standard syringes, and then connected to a retainer 10 providing connecting means to the encased needle 4 through a tip-lock 20 that will engage in the receiving means in the plunger 1. The retainer 10 is wedged into the sleeve 7 via a conically shaped collar 21. Through the force of the uncoiling compression spring 22 in the plunger 1, the needle 4 is pulled out of the sleeve 7 and retracted fully into the plunger 1 (see FIG. 3).

FIG. 3 shows the plunger 1 which houses the compression spring 22. The spring serves to retract the needle 4 when activated by the base assembly 3 (see FIG. 2) The spring 22 is compressed by means of a cylinder 23 that has a collar 19 on one end so that the spring 22 can not slide over it. The collar 19 serves as a wedge. As the spring 22 uncoils it pushes said cylinder 23 and the locked tip-lock 20 with needle 4 attached to it, to the distal end of the plunger 1. The wedged collar 19 serves two functions. One is to prevent medication from entering the plunger 1 during asperation and injection and the second is to permit the tip-lock 20 (see FIG. 2) to push the collar 19 sufficiently out of its pressure-fitted position so that the spring 22 can uncoil and pull the needle into the plunger 1.

FIG. 4 shows a plug 24, inserted into the end of the plunger 1 by means of a pressure fitting or sonic welding. It prevents the retracted needle from coming out of the back of the plunger 1 after retraction.

1. A means of permitting the user of the syringe to retract the needle into the cavity of the plunger by pushing the plunger fully forward after injection using the same hand that was used to do the injection, thereby freeing up the other hand to apply a swap to the point of injection. A unique added feature is the so-called Luer-Lock which permits exchanging of needles prior to use. While the Luer-Lock connection is not new, per se, it is unique in conjunction with a retractable needle safety syringe.
Said means comprising:
a. providing a plunger which is able to catch the base of said needle, where inter-locking means are located to permit retraction of said needle.
b. providing said needle with a set of mating locking means that will engage said plunger when said plunger is pushed all the way forward.
c. providing said base of said needle with a screw-type male adapter, commonly referred to as a luer-Lock to permit exchanging of needle sizes prior to use.
d. providing a mating female adapter to the front of said syringe to permit attaching said needles as claimed in c. above.
e. providing spring retention means that keep the spring inside said plunger in a locked position.
f. providing means that, upon providing pressure to said interlocking means (see a. above) at the front of said plunger, releases the spring, which uncoils and pulls said needle fully into said plunger.
g. providing locking means at the distal end of said plunger that render the syringe unusable after retraction.
